(19) United States
(12) Patent Application Publication
(10) Pub. No.: US 2024/0261100 A1
(43) Pub. Date: Aug. 8, 2024

(54) Minimally Invasive Repair of Heart Valve Leaflets

(71) Applicant: NeoChord, Inc., Maple Grove, MN (US)

(72) Inventors: John Zentgraf, Minneapolis, MN (US); David J. Parins, Corcoran, MN (US); Arun Saini, Burnsville, MN (US)

(21) Appl. No.: 18/640,248

(22) Filed: Apr. 19, 2024

Related U.S. Application Data
(63) Continuation of application No. 16/905,645, filed on Jun. 18, 2020, now Pat. No. 11,974,920.
Application No. 16/905,645 is a continuation of application No. 14/947,399, filed on Nov. 20, 2015, now Pat. No. 10,695,178.
Application No. 14/947,399 is a continuation of application No. 13/486,632, filed on Jun. 1, 2012, now abandoned.
(60) Provisional application No. 61/492,135, filed on Jun. 1, 2011.

(51) Int. Cl. A61F 2/24; A61B 17/00; A61B 17/04; A61B 17/06

(52) U.S. Cl. CPC A61F 2/2457; A61B 17/0057; A61B 17/0467; A61B 17/0469; A61B 17/0482; A61B 17/0487; A61B 2017/00243; A61B 2017/00606; A61B 2017/0456; A61B 2017/0496; A61B 2017/06042; A61F 2250/0012

(57) ABSTRACT

A method of repairing a heart valve provides intravascular access for repair of a heart valve through a ventricular trans-septal approach. An external guide catheter can be inserted through a vein of a patient into the right ventricle via the right atrium. An internal guide catheter can be inserted through the external guide and can provide access to the septum for a puncture tool to create an opening through the septum to the left ventricle. The internal guide can then be advanced into the left ventricle and used to guide a deployment catheter that deploys a repair device onto the heart valve.

### RELATED APPLICATION

The present application is a continuation of U.S. application Ser. No. 16/905,645 filed Jun. 18, 2020, which is a continuation of U.S. application Ser. No. 14/947,399 filed Nov. 20, 2015 which is now U.S. Pat. No. 10,695,178, which is a continuation of U.S. application Ser. No. 13/486,632 filed Jun. 1, 2012, which claims the benefit of U.S. Provisional Application No. 61/492,135 filed Jun. 1, 2011, which is incorporated herein in its entirety by reference.

### FIELD OF THE INVENTION

The present invention relates to minimally invasive delivery of a suture. More particularly, the present invention relates to attaching the suture as an artificial chordae tendineae to a flailing or prolapsing leaflet in a beating heart via an intravascular ventricular septal approach.

### BACKGROUND OF THE INVENTION

Various types of surgical procedures are currently performed to investigate, diagnose, and treat diseases of the heart and the great vessels of the thorax. Such procedures include repair and replacement of mitral, aortic, and other heart valves, repair of atrial and ventricular septal defects, pulmonary thrombectomy, treatment of aneurysms, electrophysiological mapping and ablation of the myocardium, and other procedures in which interventional devices are introduced into the interior of the heart or a great vessel.

Using current techniques, many of these procedures require a gross thoracotomy, usually in the form of a median sternotomy, to gain access into the patient's thoracic cavity. A saw or other cutting instrument is used to cut the sternum longitudinally, allowing two opposing halves of the anterior or ventral portion of the rib cage to be spread apart. A large opening into the thoracic cavity is thus created, through which the surgical team may directly visualize and operate upon the heart and other thoracic contents.

Surgical intervention within the heart by a thoracotomy generally requires isolation of the heart and coronary blood vessels from the remainder of the arterial system, and arrest of cardiac function (an “open heart” procedure). Usually, the heart is isolated from the arterial system by introducing an external aortic cross-clamp through a sternotomy and applying it to the aorta between the brachiocephalic artery and the coronary ostia. Cardioplegic fluid is then injected into the coronary arteries, either directly into the coronary ostia or through a puncture in the aortic root, so as to arrest cardiac function. In some cases, cardioplegic fluid is injected into the coronary sinus for retrograde perfusion of the myocardium. The patient is placed on cardiopulmonary bypass to maintain peripheral circulation of oxygenated blood.

Of particular interest to the present invention are open heart procedures for surgical treatment of heart valves, especially the mitral and aortic valves. According to recent estimates, more than 79,000 patients are diagnosed with aortic and mitral valve disease in U.S. hospitals each year. More than 49,000 mitral valve or aortic valve replacement procedures are performed annually in the U.S., along with a significant number of heart valve repair procedures.

Various surgical techniques may be used during an open heart procedure to repair a diseased or damaged valve, including annuloplasty (contracting the valve annulus), quadrangular resection (narrowing the valve leaflets), commissurotomy (cutting the valve commissures to separate the valve leaflets), shortening mitral or tricuspid valve chordae tendonae, reattachment of severed mitral or tricuspid valve chordae tendonae or papillary muscle tissue, and decalcification of valve and annulus tissue. Alternatively, the valve may be replaced by excising the valve leaflets of the natural valve and securing a replacement valve in the valve position, usually by suturing the replacement valve to the natural valve annulus. Various types of replacement valves are in current use, including mechanical and biological prostheses, homografts, and allografts.

The mitral valve, located between the left atrium and left ventricle of the heart, is most easily reached through the wall of the left atrium, which normally resides on the posterior side of the heart, opposite the side of the heart that is exposed by a median sternotomy. Therefore, to access the mitral valve via a sternotomy, the heart is rotated to bring the left atrium into a position accessible through the sternotomy. An opening, or atriotomy, is then made in the left atrium, anterior to the right pulmonary veins. The atriotomy is retracted by means of sutures or a retraction device, exposing the mitral valve directly posterior to the atriotomy. One of the aforementioned techniques may then be used to repair or replace the valve.

An alternative technique for mitral valve access during an open heart procedure may be used when a median sternotomy and/or rotational manipulation of the heart are/is undesirable. In this technique, a large incision is made in the right lateral side of the chest, usually in the region of the fifth intercostal space. One or more ribs may be removed from the patient, and other ribs near the incision are retracted outward to create a large opening onto the thoracic cavity. The left atrium is then exposed on the posterior side of the heart, and an atriotomy is formed in the wall of the left atrium, through which the mitral valve may be accessed for repair or replacement.

The mitral and tricuspid valves inside the human heart include an orifice (annulus), two (for the mitral) or three (for the tricuspid) leaflets and a subvalvular apparatus. The subvalvular apparatus includes multiple chordae tendineae, which connect the mobile valve leaflets to muscular structures (papillary muscles) inside the ventricles. Rupture or elongation of the chordae tendineae results in partial or generalized leaflet prolapse, which causes mitral (or tricuspid) valve regurgitation. A commonly used technique to surgically correct mitral valve regurgitation is the implantation of artificial chordae (usually 4-0 or 5-0 Gore-Tex sutures) between the prolapsing segment of the valve and the papillary muscle. This open heart operation is generally carried out through a median sternotomy and requires cardiopulmonary bypass with aortic cross-clamp and cardioplegic arrest of the heart.

Using such open heart techniques, the large opening provided by a median sternotomy or right thoracotomy enables the surgeon to see the mitral valve directly through the left atriotomy, and to position his or her hands within the thoracic cavity in close proximity to the exterior of the heart for manipulation of surgical instruments, removal of excised tissue, and/or introduction of a replacement valve through the atriotomy for attachment within the heart. However, these invasive open heart procedures produce a high degree of trauma, a significant risk of complications, an extended hospital stay, and a painful recovery period for the patient. Moreover, while heart valve surgery produces beneficial results for many patients, numerous others who might benefit from such surgery are unable or unwilling to undergo the trauma and risks of current techniques.

One alternative to open heart surgery is a robotically guided, thoracoscopically assisted cardiotomy procedure marketed under the tradename of the DaVinci® system. Instead of requiring a sternotomy, the DaVinci® system uses a minimally invasive approach guided by camera visualization and robotic techniques. Unfortunately, the DaVinci® system is not approved for mitral valve repair procedures on a beating heart. Thus, the use of the DaVinci® system for mitral valve repair still requires a cardiopulmonary bypass with aortic cross-clamp and cardioplegic arrest of the heart.

While there are other laparoscopic and minimally invasive surgical techniques and tools that have been developed, none of these devices are useable for the unique requirements of mitral valve repair on a beating heart. Suturing devices like the Superstich™ vascular suturing device or the Gore® suture passer are designed to permit manual placement of sutures as part of a surgical procedure, but are not designed for use on a beating heart. While certain annuloplasty techniques and instruments that can suture an annuloplasty ring as part of vascular repair or heart bypass surgery may be used in conjunction with a beating heart, these annuloplasty procedures do not involve the capture or retention of a constantly moving leaflet. Consequently, the design and use of annuloplasty techniques and instruments are of little help in solving the problems of developing instruments and techniques for minimally invasive thoracoscopic repair of heart valves during a beating heart procedure.

Recently, a technique has been developed for minimally invasive thoracoscopic repair of heart valves while the heart is still beating. Int'l Pub. No. WO 2006/078694 A2 to Speziali, which is incorporated by reference herein, discloses a thoracoscopic heart valve repair method and apparatus. Instead of requiring open heart surgery on a stopped heart, the thorascopic heart valve repair methods and apparatus taught by Speziali utilize fiber optic technology in conjunction with transesophageal echocardiography (TEE) as a visualization technique during a minimally invasive surgical procedure that can be utilized on a beating heart. U.S. Publication No. 2008/0228223 to Alkhatib also discloses a similar apparatus for attaching a prosthetic tether between a leaflet of a patient's heart valve and another portion of the patient's heart to help prevent prolapse of the leaflet and/or to otherwise improve leaflet function.

More recent versions of these techniques are disclosed in U.S. Patent Application Publication Nos. 2009/0105751 and 2009/0105729 to Zentgraf, which disclose an integrated device that can enter the heart chamber, navigate to the leaflet, capture the leaflet, confirm proper capture, and deliver a suture as part of a mitral valve regurgitation (MR) repair.

These references disclose suturing valve leaflets by accessing the heart through an open surgical approach that requires an artificial opening in the heart wall be made, for example at the apex of the ventricle, during the open surgical approach. It would be advantageous for a minimally invasive suture delivery system to be able to suture valve leaflets in a beating heart procedure without requiring an open surgical approach or an incision into the exterior ventricular wall in order to minimize blood loss.

### SUMMARY OF THE INVENTION

Embodiments of the present invention allow for repair of heart valve regurgitation during a beating heart procedure including various steps and apparatuses for entering the heart chamber, navigating to a heart valve leaflet, capturing the leaflet, confirming proper capture, and delivering a suture. The devices and procedures of these embodiments can be used with an intravascular catheter based approach for delivery of sutures for the treatment of heart valve regurgitation.

In one embodiment, the system provides venous access into a heart chamber (venous access via the femoral or jugular vein) while minimizing the loss of blood within and without the system. The device can be inserted through the right atrium and into the right ventricle, with the position within the ventricular apex visualized via ultrasound or fluoroscopy. Once access into the heart chamber is achieved, the system is positioned via a non-invasive imaging modality. The system allows capture of intra-cardiac tissue structure. Once captured, the system allows control to be maintained over said tissue structure. Imaging modalities allow confirmation of proper capture position of the system relative to the tissue structure. The system then accommodates the delivery of the deployment catheter to said tissue structure once proper position has been confirmed.

In one embodiment, a guide-in-guide catheter system provides venous access to the ventricular septal wall for a trans-septal puncture tool to provide the access to the left ventricular cavity. Once the left ventricle is accessed, an internal guide catheter can be advanced within the external guide across the septal wall into the left ventricle. The external guide catheter can have a side exiting lumen to facilitate the positioning of the internal guide, or alternatively a septal puncture catheter with a septal puncture device therein, to the selected area for crossing the ventricular septum. A curve in the guide can angle the tip of the catheter to the desired location for trans-septal puncture. A guide wire may be used to maintain position. After the septal puncture is completed the device can be removed and a dilator inserted into the internal guide to aid the passage of the guide through the septal wall. The dilator can be removed after the internal guide has crossed the septal wall. The internal guide can also have a pre-shaped curvature to the distal tip. This curve can provide the direction support to guide the deployment catheter toward the mitral valve.

The deployment catheter can have a central lumen to accept a guide wire used in positioning the deployment catheter to effectively engage the mitral valve. The central lumen can also be used for an intravascular ultrasound device or a direct visualization device. The suture is deployed by the deployment catheter at the selected site. The deployment catheter can be withdrawn from the guide catheter and re-loaded or replaced for successive suture deployments.

In one embodiment, a medical repair device may be added to the procedure, such as a leaflet extension, a passive valve occlusion device or a pledget. The deployed sutures exit the internal guide catheter and can be temporarily fixed outside the body. Once the desired amount of sutures is positioned, they can be loaded through a central lumen of a septal seal device. The septal seal device is advanced through the external guide catheter and guided, via the sutures and external guide catheter, through the ventricular puncture site. The right ventricular side of the seal device is deployed and then the left side of the seal device is deployed. The internal catheter is then detached from the septal seal element and withdrawn from the external guide catheter. The sutures remain in the internal lumen of the septal closure device attached to the mitral valve and exit through the external guide.

In one embodiment, the sutures can have the tension individually adjusted to evaluate the physiological effect. The evaluation can be done using transesophageal echocardiography or other non-invasive methods. If the suture is overly tightened, a catheter can be delivered through the external guide to the lumen seal inside of the septal seal device. Advancing the catheter through the seal will release suture tension and allow for re-tensioning. When the tensioning task is complete, the sutures can be fixed at the septal seal element.

In one embodiment, an anchor catheter with a distally mounted cam lock element or other mechanical lock permanently fixes to the septal seal element and fixes the position of the sutures while maintaining the adjusted tension. This step completes the septal seal and suture tensioning. The anchor catheter can then be withdrawn with the proximal ends of the sutures. The sutures can then be threaded through the lumen or opening of a cutting catheter. A cutting catheter can be advanced over the sutures until it contacts the septal seal device. The cutting catheter then cuts the sutures at the seal to complete the implant procedure. The entire catheter system is then removed from the patient and the access site closed.

In another embodiment, a deployment catheter is capable of multiple suture deployments in a single activation. This would reduce the number of instrument exchanges and provide increased control of the position of the sutures relative to each other.

A further embodiment uses the sutures to deliver a biomatrix patch to enhance closure. The patch can be attached to the valve with the sutures. The patch could be delivered to either the ventricular or atrial side of the mitral valve leaflet. This patch can improve leaflet coaptation and reduce/eliminate mitral valve regurgitation by augmenting the native leaflet tissue structure supported by the delivery of a biomatrix material that can support the mitral valve annular ring or subvalvular apparatus.

Another embodiment includes the deployment of a passive occlusive device intended to improve valve closure, the device would be delivered, positioned, and anchored via the ventricular septal approach described herein.

The above summary of the various embodiments of the invention is not intended to describe each illustrated embodiment or every implementation of the invention. This summary represents a simplified overview of certain aspects of the invention to facilitate a basic understanding of the invention and is not intended to identify key or critical elements of the invention or delineate the scope of the invention.

### BRIEF DESCRIPTION OF DRAWINGS

The embodiments of the present invention may be more completely understood in consideration of the following detailed description of various embodiments in connection with the accompanying drawings, in which:

FIG. 1 is a view of a device for venous access into a heart chamber via the femoral vein to facilitate repair of a heart valve leaflet according to an embodiment of the present invention;

FIG. 2A is a view of a valve leaflet repair device according to an embodiment of the present invention with an internal guide and puncture tool passed into the left ventricle;

FIG. 2B is a partial view of the valve leaflet repair device depicted in FIG. 2A;

FIG. 3 is a view of a valve leaflet repair device according to an embodiment of the present invention with an internal guide exiting a side exit guide catheter;

FIG. 4 is a view of a valve leaflet repair device according to an embodiment of the present invention with a deployment catheter exiting an internal guide and positioned at the mitral valve;

FIG. 5A is a view of a deployment catheter tip according to an embodiment of the present invention with a moveable catheter jaw and a suture capture needle, with the catheter jaw in the closed position;

FIG. 5B is a view of the deployment catheter tip of FIG. 5A with the moveable catheter in the open position;

FIG. 6 is a cross-sectional view of the deployment catheter tip of FIGS. 5A and 5B;

FIG. 7 is a view of a valve leaflet repair device according to an embodiment of the present invention with several sutures attached to the mitral valve and exiting through an internal guide;

FIG. 8 is a view of a valve leaflet repair device according to an embodiment of the present invention with ventricular septal seal devices deployed in the septal wall with sutures extending through a center lumen;

FIG. 9 is a schematic representation of the septal seal device of FIG. 8 in place in the heart;

FIG. 10 is a perspective view of septal seal types according to embodiments of the present invention;

FIG. 11 is a partial view of a septal seal device lumen with a seal element for holding a suture in tension showing the suture freed from tension by a catheter that releases the seal element;

FIG. 12 is a view of a valve leaflet repair device according to an embodiment of the present invention with an anchor device fixing the position of the sutures;

FIG. 13 is a side cut-away view of the anchor device of FIG. 12 having a fixation catheter with a locking element for mating with seal internal lock features;

FIG. 14 is a view of a valve leaflet repair device according to an embodiment of the present invention with a cutting device for cutting sutures at the right ventricular side of a septal seal;

FIG. 15 is a side cross-sectional view of the suture cutting device of FIG. 14;

FIG. 16 is a view of a completed implant procedure using a valve leaflet repair device according to an embodiment of the present invention;

FIG. 17 is a flow chart of surgical procedural steps for repair of heart valve leaflets according to an embodiment of the present invention.

While the present invention is amenable to various modifications and alternative forms, specifics thereof have been shown by way of example in the drawings and will be described in detail. It should be understood, however, that the intention is not to limit the present invention to the particular embodiments described. On the contrary, the invention is to cover all modifications, equivalents, and alternatives falling within the spirit and scope of the present invention.

### DESCRIPTION OF EMBODIMENTS

In the following detailed description of the present invention, numerous specific details are set forth in order to provide a thorough understanding of the present invention. However, one skilled in the art will recognize that various embodiments of the present invention may be practiced without these specific details. In other instances, well-known methods, procedures, and components have not been described in detail so as to not unnecessarily obscure aspects of the present invention.

One embodiment of the heart valve repair and delivery system will be examined to demonstrate the multiple catheter access steps required to enter the target heart chamber and deliver the repair device. This embodiment performs the repair of mitral valve regurgitation by delivering sutures to repair the defective valve with a deployment catheter that acts to reduce/eliminate mitral valve regurgitation (MR). In other embodiments, the access approach described herein can be used to access the heart for any other type of procedure, such as, for example, a heart valve replacement, repair of another heart structure or delivery of repair devices other than sutures to valve leaflets.

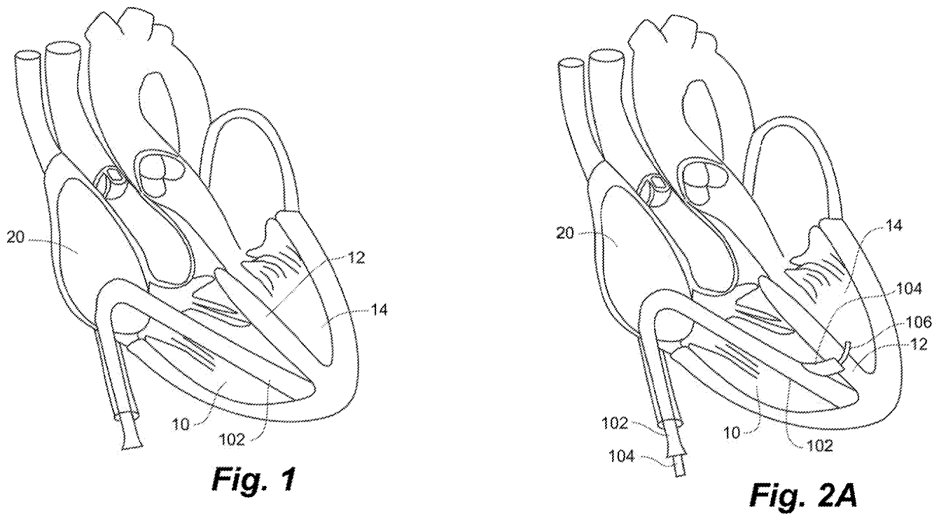

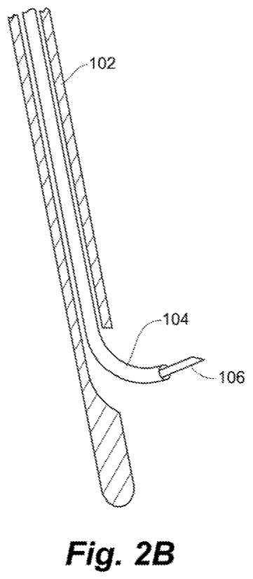

Embodiments of the present invention can be used as a vascular access system. It can include a standard vascular introducer that 1) eliminates the need for multiple passes of the instrument against the vein wall, 2) minimizes blood loss due to instrument leakage (circular components are more amenable to closer tolerances and sealing capability), and 3) reduces push/pull forces on the vein wall. The introducer contains seals to maintain hemostasis during instrument exchanges. A side exiting external guide catheter 102 can provide access into the right ventricle 10 as shown in FIG. 1. In one embodiment, a distal end of the external guide 102 can include a suction element to ensure that it holds its position in the right ventricle at, for example, the right ventricular apex. The system can include an internal guide catheter 104 disposed in the side exiting external guide catheter 102 design that facilitates the access through the right ventricle 10 to the right ventricular wall. The introducer and/or external guide catheter 102 can therefore function as means for accessing the right ventricle. A standard septal puncture tool 106 with a needle like end can serve as a means for creating an opening in the septum to create the hole in the ventricular septal wall 12 to provide the passageway for the guide catheter 104 through the wall as depicted in FIG. 2A. As used herein, the term catheter can refer to an elongate, generally flexible and tubular medical device that extends along a longitudinal axis and defines a diameter around the longitudinal axis.

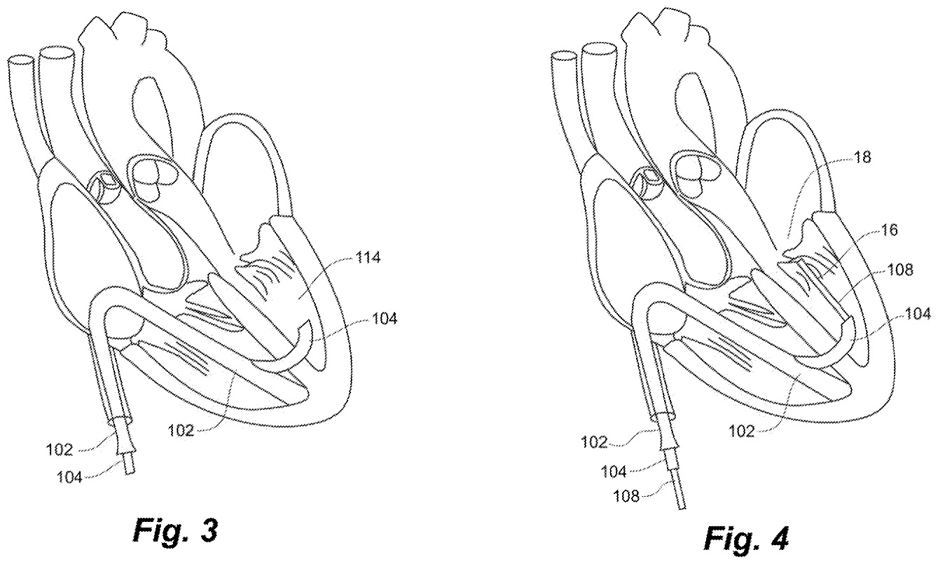

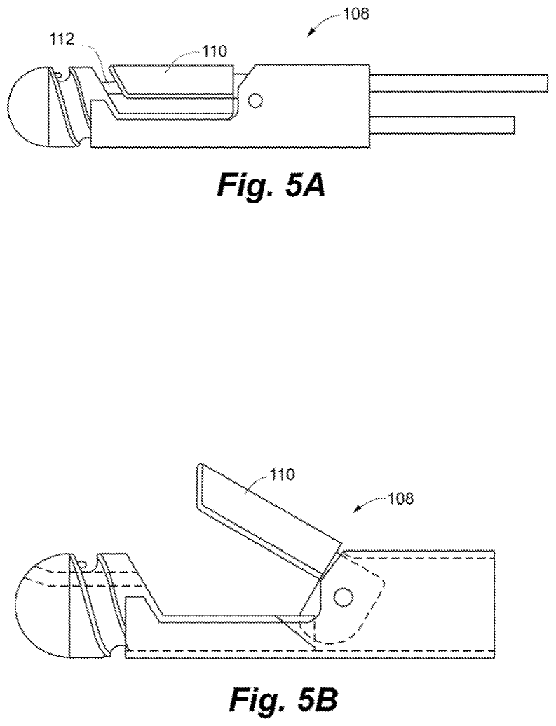

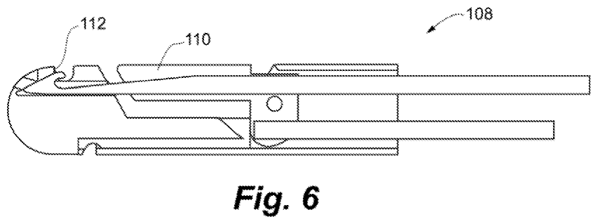

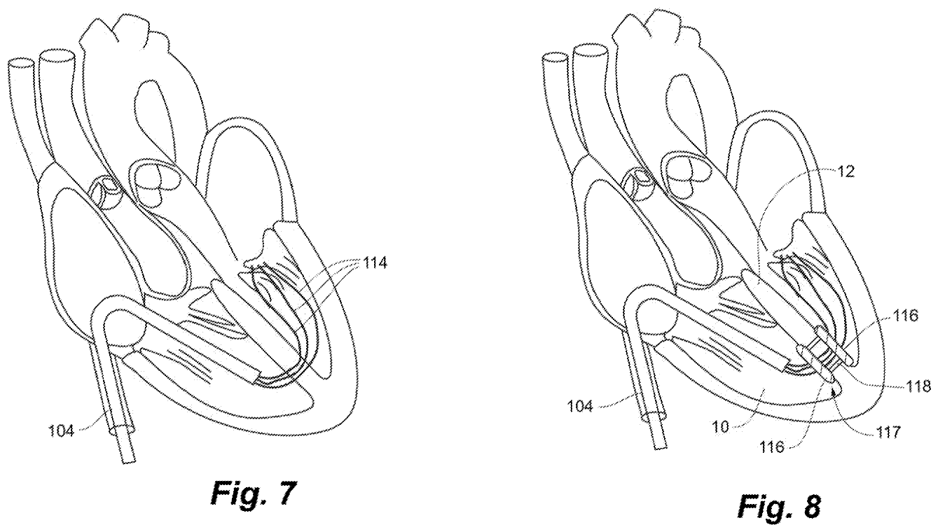

The pre-shaped internal guide catheter 104 is then advanced into the left ventricle 14, as shown in FIG. 3, and positioned to deliver a deployment catheter 108 to properly capture a leaflet 16 of the mitral valve 18 for repair as shown in FIG. 4. The internal guide catheter 104 can therefore function as a means for positioning the deployment catheter 108 in the left ventricle. The deployment catheter 108, as shown in FIGS. 5A-5B and 6, can provide a means for deploying a repair device and can include a clamping mechanism 110 or other means for grasping for capturing the leaflet and a suture deployment mechanism including a suture capture needle 112 or other means for inserting the suture into the leaflet. The deployment catheter 108 can be exchangeable within the guide catheter 104 to permit multiple suture 114 deployments on the valve leaflet as shown in FIG. 7. Alternatively, the deployment catheter 108 can deliver several sutures 114 at one deployment. Note that in some Figures, such as FIG. 7, the external guide catheter 102 is not shown for sake of clarity.

As can be seen in FIG. 4, embodiments of the present invention provide a tri-catheter approach for accessing a heart valve to deploy a repair device onto a portion of the valve, such as a valve leaflet. The tri-catheter approach can include the external guide catheter 102, internal guide catheter 104 received within the external guide catheter 102 and deployment catheter 108 received within the internal guide catheter 104. In some embodiments, as depicted in FIG. 4, the tri-catheter arrangement can define a generally S-shaped access configuration to the valve with the catheters defining a first curve in the right atrium to access the right ventricle and a second curve where the internal guide 104 exits the external guide 102 to cross the septum and access the heart valve in the left ventricle. In one embodiment, the external guide 102 defines a curvature of about 130 degrees and the internal guide 104 has a generally U-shaped distal end that angles towards the valve to define the generally S-shaped configuration. Both external guide 102 and internal guide 104 may be given various curvatures to match the anatomy of a given patient. In one embodiment, the external guide 102 has a diameter of between 12 and 16 French and the inner guide 104 has approximately 2 French sizes smaller than the external guide 102. The delivery catheter 108 and other catheters inserted into the internal guide 104 can have a diameter that is approximately 2 French sizes smaller than the internal guide 104.

The deployment catheter 108 can alternatively or additionally deliver an additional medical repair device such as a leaflet extension or a passive valve occlusion device. A medical repair device is a device that is permanently implanted for the repair treatment or a device that supports the primary repair treatment. Such medical repair devices can be suture materials, biomatrix materials used to support or augment a tissue structure, or devices that would provide repair treatment by device assisted coaptation of one of the cardiac valves. In one embodiment, deployment catheter 108 can deliver a pledget, such as described in commonly owned, copending U.S. patent application Ser. No. 13/339,865, which is incorporated by reference herein. In another embodiment, deployment catheter 108 can deliver a replacement valve or a device that seats in the valve annulus and has a portion extending down between the valve leaflets that is anchored to the heart.

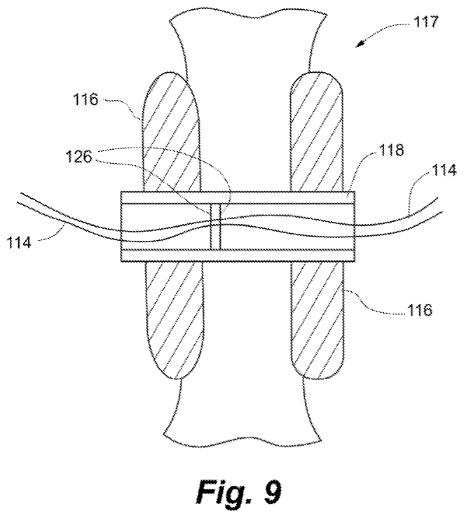

After the desired number of sutures 114 is deployed, the sutures 114 are threaded through a lumen of a septal seal device 117. The septal sealing device 117 is then advanced down the guide catheter 104 with a seal catheter and into the right ventricle 10. The device 117 is positioned to have right side and left side seal elements 116, depicted in FIG. 9, positioned on opposite sides of the septal wall 12. The sealing elements 116 are deployed to provide a means for sealing the opening in the septum with the sealing device 117 and the catheter withdrawn as shown in FIG. 8. In one embodiment, seal device 117 comprise a pre-shaped wire frame having tensioned flanges on opposing sides that abut the opposing sides of the septal wall 12 to hold the seal elements 116 in place and an internal lumen 118 extending through the device. In one embodiment, the wire frame is comprised of Nitinol.

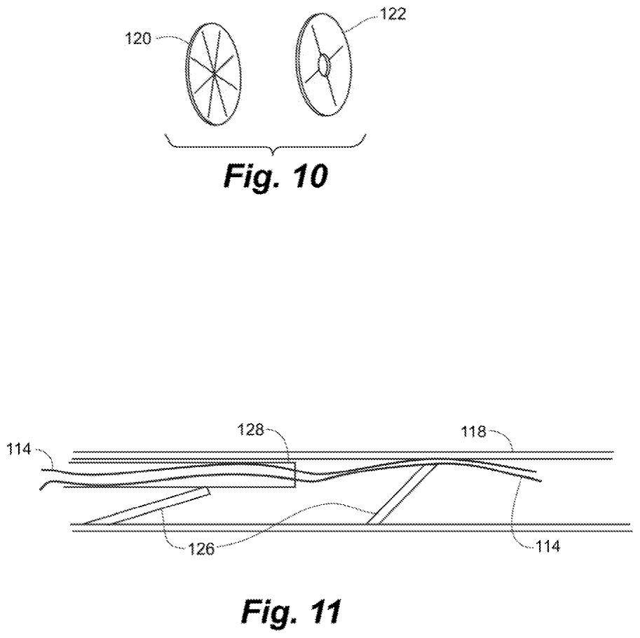

The sutures 114 can now be tensioned from a location external of the heart to have a desired tension that provides for proper valve function. The internal lumen 118 of the septal sealing device 117 can have one or more seals 126 that provide pressure on the sutures to prevent them from easily moving to maintain the set tension on the sutures 114 and provide a means for setting the tension. Seals 126 can also serve to maintain the integrity of the lumen 118. The seal can be similar to a silicone slit seal 122 or a flap seal 120, as shown in FIG. 10, both of which facilitate release of the suture 114 position using a catheter 128 or other means for re-tensioning if desired to allow for re-tensioning, as shown in FIG. 11.

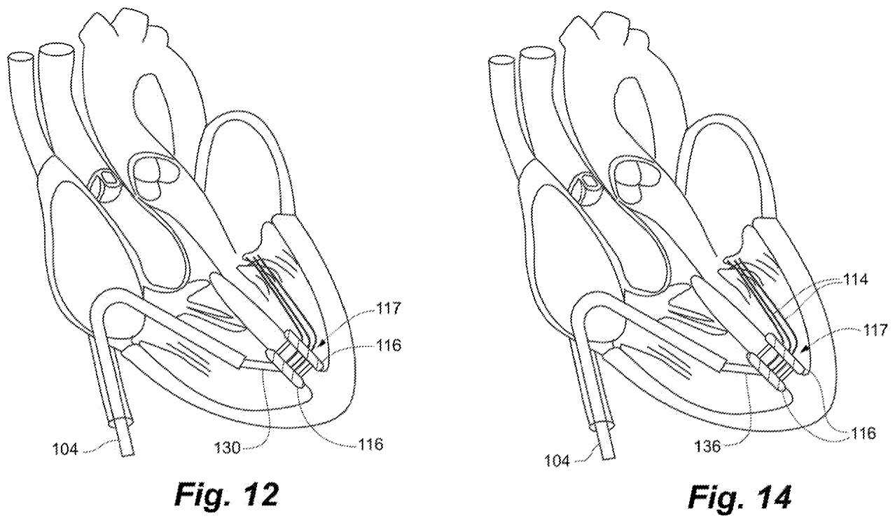

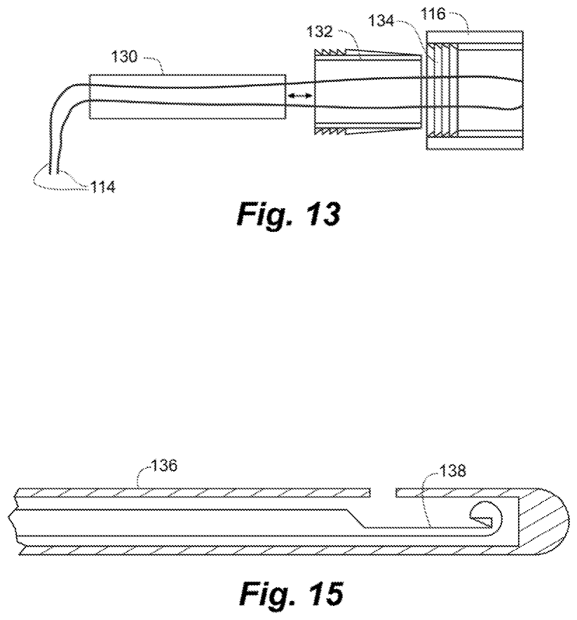

After tension of the sutures 114 is confirmed via trans-esophageal echo cardiography, for example, the sutures 114 can be fixed to the sealing device 117 for permanent anchoring of the sutures 114. The sutures 114 are threaded through a lumen in an anchoring catheter 130 to provide coaxial positioning of a locking element 132 or anchoring device that can function as a means for anchoring the sutures at the sealing device 117. Fixation can be accomplished with the anchoring catheter 130 with the releasable locking element 132 that interfaces with internal lock features 134 in the right side sealing element 116 of the sealing device 117 and locks the sutures 114 in position and permanently fixes to the sealing device 117 as shown in FIGS. 12 and 13. The locking mechanism 132 can be a rotational cam lock or a screw in element.

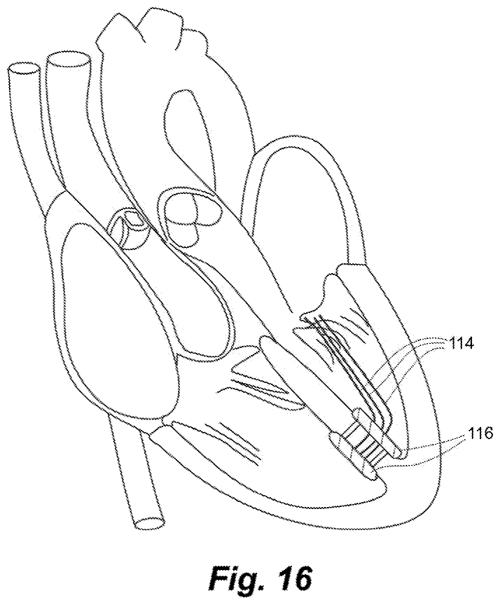

Once the sutures 114 are permanently fixed to the sealing element 116, the sutures 114 can be threaded through the end of a cutting catheter 136 which is advanced until it contacts the sealing element 116 as shown in FIG. 14. The sutures 114 can then be cut at the sealing element 116 with a cutting device or tool 138 in the cutting catheter 136, also shown in FIG. 15, which is then withdrawn. The intervention is then complete and the guide catheters and introducers can be withdrawn leaving behind the anchored sutures 114 as shown in FIG. 16. The access site can then be closed.

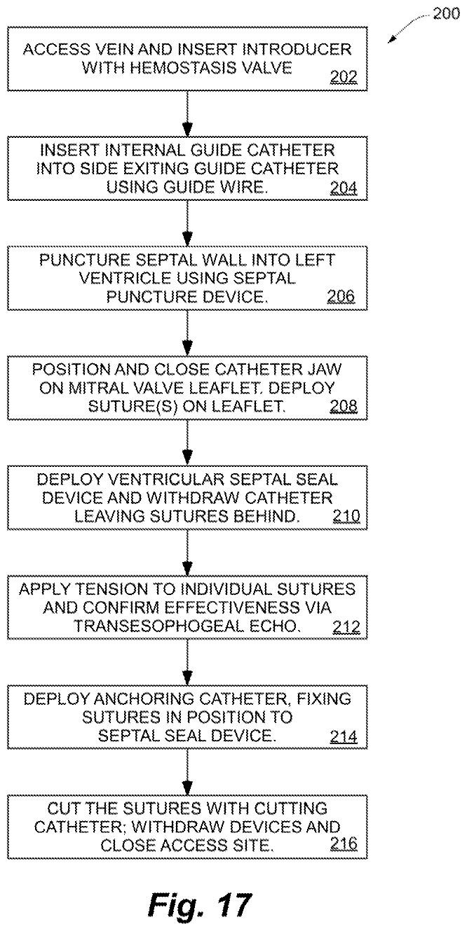

FIG. 17 depicts a flowchart of surgical steps 200 that can be taken to repair a heart valve leaflet according to an embodiment of the present invention. At step 202, the femoral or jugular vein is accessed via a cut down or Seldinger technique and an introducer with a hemostasis valve is inserted into the vein. In one embodiment, the outer diameter of the introducer is a maximum of 24 french. At step 202, access is gained to the right atrium 20 using a guide wire and an external guide catheter 102 is advanced over the guide wire to the ventricular apex. In one embodiment, the external guide catheter 102 is a side-exiting catheter. An internal guide catheter 104 is inserted into the external guide catheter following removal of the guide wire until it exits the external guide. At step 206, proper positioning of the internal guide catheter for puncture of the ventricular septal wall 12 is confirmed and a septal puncture device 106 is inserted into the internal guide 104 and advanced to the desired position at the septal wall 12 to puncture the septal wall 12. A guide wire can then be advanced through the internal guide 104 to maintain position in the left ventricle 14. The puncture tool 106 can be withdrawn and a dilator can be used to facilitate passage of the internal guide catheter 104 into the left ventricle 14 and then withdrawn.

At step 208, a suture deployment catheter 108 can be inserted into the internal guide catheter 104 and advanced in the left ventricle 14. The deployment catheter 108 can be positioned near the leaflet 16, capture the leaflet 16 with a moveable jaw 110, advance a suture needle 112 through the leaflet 16, withdraw the needle 112 back through the leaflet 16 and into the catheter 108, release the leaflet 16 and be withdrawn. In one embodiment, proper capture of the valve leaflet 16 is confirmed prior to advancing the needle 112 through the leaflet 16. In one embodiment, this can be done with a fiber optic visualization system. In one embodiment, deployment catheter 108 can be reinserted to deploy additional sutures 114 onto leaflet 16. In another embodiment, leaflet capture and suture deployment can be aided with an augmented reality navigation system utilizing magnetic tracking such as is disclosed in commonly owned, copending U.S. Provisional Application No. 61/565,795, which is hereby incorporated by reference. In some embodiments, deployment catheter 108 can deploy multiple sutures 114 onto leaflet 16 in a single insertion.

At step 210, the sutures 114 are threaded through a lumen 118 of a ventricular septal sealing device 117, which is then advanced to the ventricular septal wall 12 puncture site with a septal sealing catheter. The septal seal device 117 can have seal elements 116 deployed to seal the puncture and the septal sealing catheter is withdrawn, leaving the sutures 114 in the sealing device 117 and extending outward through the body. At step 212, the sutures 114 can be tensioned to a desired level for proper valve leaflet function. In one embodiment, proper tensioning of sutures 114 and valve leaflet function can be confirmed via transesophogeal echo. In one embodiment, tension of the sutures 114 can be released using a catheter 128 and readjusted.

At step 214, the sutures 114 are inserted into a lumen of an anchoring catheter 130, which is advanced through the internal guide 104 to the septal sealing device 117. An anchoring element 132 can then be deployed into the sealing device 117 to fix the sutures 114 in position in the sealing device 117 and the anchoring catheter 130 can be withdrawn. At step 216, a suture cutting catheter 136 is inserted into the guide catheter and used to cut the sutures adjacent the septal sealing device 117 with a cutting element 138. The cutting catheter 136, guide catheters 102, 104 and introducers can then all be withdrawn and the access site can be closed to complete the procedure.

Although the system and method described herein are primarily described in connection with intravenous access for a ventricular septal approach, it should be understood that the devices and methods described can be adapted for use with various other approaches. For example, the system can also provide venous access to the atrial septal wall for a trans-septal puncture that provides access to the left atrium. In addition, the system can be used to provide venous access to the left ventricle through the aortic valve.

It should further be noted that although the system and method described herein are primarily described with reference to repairing a heart valve leaflet, other tissue structures can be targeted for repair as well. For example, the papillary muscle, heart wall or any other intra-cardiac structure can be targeted for repair or anchoring.

In various embodiments, a heart valve repair system as described herein can be provided as a kit including the various catheters and devices described herein and instructions for repairing a heart valve of a patient as described herein. In one embodiment, the present application comprises the instructions. In another embodiment, an FDA required Instructions for Use can comprise the instructions.

Various embodiments of systems, devices, and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

1. A method of repairing a heart valve in a beating heart of a patient, comprising:
inserting a guide catheter through a vein of a patient and into a right atrium of a heart of the patient;
inserting a septal puncture tool through the guide catheter and into the heart; puncturing a septum in the patient's heart to create an opening through the septum;
advancing a deployment catheter through the guide catheter and through the septum to a position adjacent a mitral valve leaflet;
inserting a suture through the mitral valve leaflet with the deployment catheter; inserting an anchor catheter through the guide catheter to insert an anchor device adjacent a left ventricle of the heart such that the suture extends from the mitral valve leaflet to the anchor device;
adjusting a tension of the suture to achieve proper valve function; and
locking the suture at the anchor device at the tension that achieves proper valve function.

2. The method of claim 1, further comprising withdrawing the anchor catheter from the body with the mechanical lock anchor device engaged with the suture in the heart.

3. The method of claim 1, further comprising inserting the suture through the anchor device prior to inserting the anchor catheter into the body.

4. The method of claim 1, wherein inserting the anchor device adjacent the left ventricle includes advancing the anchor device along the suture to adjacent the left ventricle.

5. The method of claim 1, further comprising inserting a cutting catheter having a cutting tool through the guide catheter to adjacent the anchor device after adjusting the tension of the suture and cutting the suture adjacent the anchor device with the cutting tool.

6. The method of claim 1, wherein the step of inserting a suture through the mitral valve leaflet with the deployment catheter includes:
capturing a heart valve leaflet with a clamping mechanism of the deployment catheter; and
inserting the suture through the heart valve leaflet with a needle of the deployment catheter.

7. A method of repairing a heart valve in a beating heart of a patient comprising:
intravenously advancing a deployment catheter into a left ventricle of a beating heart of a patient through a septum in the heart;
inserting a suture through a mitral valve leaflet with the deployment catheter;
inserting an anchor catheter into the heart to insert an anchor device adjacent the left ventricle such that the suture extends from the mitral valve leaflet to the anchor device;
adjusting a tension of the suture to achieve proper valve function; and
locking the suture at the anchor device at the tension that achieves proper valve function.

8. The method of claim 7, further comprising withdrawing the anchor catheter from the body with the mechanical lock anchor device engaged with the suture in the heart.

9. The method of claim 7, further comprising inserting the suture through the anchor device prior to inserting the anchor catheter into the body.

10. The method of claim 7, wherein inserting the anchor device adjacent the left ventricle includes advancing the anchor device along the suture to adjacent the left ventricle.

11. The method of claim 7, further comprising inserting a cutting catheter having a cutting tool into the heart adjacent the anchor device after adjusting the tension of the suture and cutting the suture adjacent the anchor device with the cutting tool.

12. The method of claim 7, wherein the step of inserting a suture through the mitral valve leaflet with the deployment catheter includes:
capturing a heart valve leaflet with a clamping mechanism of the deployment catheter; and
inserting the suture through the heart valve leaflet with a needle of the deployment catheter.
